(19) United States
(12) Patent Application Publication
(10) Pub. No.: US 2011/0111974 A1
(43) Pub. Date: May 12, 2011

(54) Short Duplex Probes for Enhanced Target Hybridization

(75) Inventors: CHAD A. MIRKIN, WILMETTE, IL (US); ANDREW E. PRIGODICH, EVANSTON, IL (US); DWIGHT S. SEFEROS, TORONTO (CA)

(73) Assignee: NORTHWESTERN UNIVERSITY, EVANSTON, IL (US)

(21) Appl. No.: 12/911,581

(22) Filed: Oct. 25, 2010

Related U.S. Application Data
(60) Provisional application No. 61/254,566, filed on Oct. 23, 2009.
(60) Provisional application No. 61/316,707, filed on Mar. 23, 2010.

(51) Int. Cl. C40B 30/04; C40B 40/06; C12N 5/071

(52) U.S. Cl. 506/9; 506/16; 435/375

(57) ABSTRACT

The present disclosure is directed to compositions and methods for detecting or associating with a target polynucleotide.

### CROSS REFERENCE TO RELATED APPLICATIONS

This application claims the priority benefit under 35 U.S.C. §119(e) of U.S. Provisional Application No. 61/254,566, filed Oct. 23, 2009 and U.S. Provisional Application No. 61/316,707, filed Mar. 23, 2010, the disclosures of which are incorporated herein by reference in their entirety.

### STATEMENT OF GOVERNMENT INTEREST

This invention was made with government support under grant numbers 5U54-CA 119341, awarded by the National Institutes of Health (NCI/CCNE) and 5DP1 OD000285, awarded from a NIH Director's Pioneer Award, grant number EEC-0647560, awarded by the NSF/NSEC, and grant number N00244-09-1-0071, awarded by a National Security Science and Engineering Faculty (NSSEF) Fellowship. The government has certain rights in the invention.

### FIELD OF THE INVENTION

The present disclosure is directed to compositions and methods for detecting or associating with a target polynucleotide.

### BACKGROUND OF THE INVENTION

Methods to increase the rate of DNA hybridization on surfaces in order to improve the speed and efficiency of bioinformatic assays, diagnostics and therapeutic agents [Wang et al., Angew. Chem., Int. Ed. 48: 856-870 (2009); Katz et al., Angewandte Chemie-International Edition 43: 6042-6108 (2004); Rosi et al., Chemical Reviews 105: 1547-1562 (2005); Simmel et al., Small 1: 284-299 (2005); Bath et al., Nature Nanotechnology 2: 275-284 (2007)] are needed. Such methods should be easily employed, compatible with a wide range of sequences and retain activity both inside and outside cells. Previous approaches to increase DNA hybridization rates included the use of locked nucleic acids (LNAs), hairpin disruption and the incorporation of a region of double-stranded DNA (dsDNA) adjacent to a single-stranded DNA (ssDNA) target hybridization site. “Designer” DNA analogs such as LNA rapidly hybridize target DNA, but the relatively high cost of these nucleic acids has prevented their widespread use [Wang et al., J. Am. Chem. Soc. 127: 15664-15665 (2005); Castoldi et al., RNA-A Publication of the RNA Society 12, 913-920 (2006); Martinez et al., Analytical Chemistry 81, 3448-3454 (2009)]. Hairpin disruption increases interstrand DNA binding rates by using an additional DNA molecule to block the competing intrastrand hybridization [Seelig et al., Journal of the American Chemical Society 128: 12211-12220 (2006); Wei et al., Nucleic Acids Research 36: 2926-2938 (2008); Gao et al., Nucleic Acids Research 34: 3370-3377 (2006); Zhang et al., Journal of the American Chemical Society 131: 17303-17314 (2009); Wang et al., Physical Review E 72: 051918 (2005); Leunissen et al., Nature Materials 8: 590-595 (2009); Dreyfus et al., Physical Review Letters 102: 048301 (2009)]. This approach is only useful when the sequence of interest naturally hairpins, making it incompatible with applications that require the use of a broad range of sequences. Another approach for increasing binding rates is the use of a region of dsDNA adjacent to a ssDNA target hybridization site [Maye et al., Journal of the American Chemical Society 128: 14020-14021 (2006); Riccelli et al., Nucleic Acids Research 29: 996-1004 (2001); O'Meara et al., Analytical Biochemistry 255: 195-203 (1998)]. The second duplex creates an additional base-stacking interaction with the incoming target, thermodynamically stabilizing hybridization. However, this approach predominantly affects the thermodynamics, not the kinetics, and has not been demonstrated in an intracellular environment [Yuan et al., Chemical Communications 6600-6602 (2008); Vasiliskov et al., Nucleic Acids Research 29: 2303-2313 (2001)]. It has also been proposed that structural changes caused by the dsDNA region could increase target hybridization kinetics on the surface of a nanoparticle [Maye et al., J. Am. Chem. Soc. 128: 14020-14021 (2006)]. However, previous work in this area has been performed on materials that allow both structural changes and base-stacking interactions to occur, making it difficult to experimentally distinguish the two factors [Riccelli et al., Nucleic Acids Res 29: 996-1004 (2001); O'Meara et al., Anal. Biochem 255: 195-203 (1998); Maye et al., J Am Chem Soc 128: 14020-14021 (2006)]. In addition to questions about the mechanism of action, the adjacent duplex strategy has several limitations. It has not been used to selectively “turn on” the hybridization of a specific sequence in a solution of many targets and capture sequences, and it is poorly suited for in situ biological applications. As such, the need remains for a general approach to dynamically control the rate of DNA hybridization both in and outside of cells.

One class of materials where DNA hybridization is particularly important is DNA functionalized gold nanoparticles (DNA-Au NPs), which consist of a spherical gold core with a dense monolayer of DNA covalently bound to the gold surface [Mirkin et al., Nature 382: 607-609 (1996)]. The unique architecture of DNA-Au NPs results in cooperative hybridization [Lytton-Jean et al., J. Am. Chem. Soc. 127: 12754-12755 (2005)], resistance to nucleases [Seferos et al., Nano Lett. 9: 308-311 (2009)], and extraordinary cellular uptake [Giljohann et al., Nano Lett. 7: 3818-3821 (2007)]. This combination of hybridization and cellular properties has proven useful in materials self-assembly [Mirkin et al., Nature 382: 607-609 (1996); Alivisatos et al., Nature 382: 609-611 (1996); Park et al., Nature 451: 553-556 (2008)], extracellular diagnostics [Elghanian et al., Science 277: 1078-1081 (1997); Park et al., Science 295: 1503-1506 (2002)], intracellular biodetection [Seferos et al., J. Am. Chem. Soc. 129: 15477-15479 (2007); Zheng et al., Nano Letters 9: 3258-3261 (2009); Prigodich et al., Acs Nano 3: 2147-2152 (2009)] and gene regulation [Rosi et al., Science 312: 1027-1030 (2006); Patel et al., Proc. Natl. Acad. Sci. U.S.A. 105: 17222-17226 (2008); Giljohann et al., Journal of the American Chemical Society 131: 2072-2073 (2009)]. However, the kinetics of target hybridization to DNA-Au NPs are still not fully understood [Chen et al., Nucleic Acids Res 37(11): 3756-65 (2009)].

### SUMMARY OF THE INVENTION

The present disclosure provides compositions and methods for increasing the rate of polynucleotide hybridization at surfaces using a type of polynucleotide architecture. The rate enhancement involves a structural change in the polynucleotide that moves the single stranded polynucleotide binding domain away from a surface, making it more available to an incoming target polynucleotide (FIG. 9). The structural change is isolated to a short internal complementary polynucleotide (sicPN) bound polynucleotide, and an additional aspect of the disclosure involves a plurality of surface-functionalized polynucleotides, and a plurality of sicPNs.

Accordingly, a composition provided by the disclosure comprises a surface functionalized with a plurality of polynucleotides, each polynucleotide in the plurality functionalized to the surface at a terminus of the polynucleotide, the composition further comprising a plurality of short internal complementary polynucleotides (sicPNs) having a sequence sufficiently complementary to a portion of each polynucleotide in the plurality such that under appropriate conditions, a sicPN in the plurality of sicPNs is able to associate with each polynucleotide over a portion of each polynucleotide, the portion of each polynucleotide located proximal to the terminus of each polynucleotide that is functionalized to the surface, each polynucleotide having a length longer than each sicPN in the plurality to provide a single stranded portion of each polynucleotide when a polynucleotide in the plurality is associated with a sicPN in the plurality of sicPNs, the single stranded portion of the polynucleotide located distal to the portion of the polynucleotide to which the sicPN associates, the single stranded portion having a sequence sufficiently complementary to a target polynucleotide to associate with the target polynucleotide under appropriate conditions, wherein association of the polynucleotide with the target polynucleotide displaces and/or releases the sicPN associated with the polynucleotide, and wherein at least 25% of all polynucleotides in the plurality are associated with a sicPN. In some aspects, at least 75% of all polynucleotides are associated with a sicPN. The surface to which the plurality of polynucleotides is functionalized is, in various aspects, a nanoparticle or a solid support, and in a further aspect the solid support is a microarray. In some embodiments, association of the polynucleotide with the target polynucleotide displaces and/or releases a sicPN associated with the polynucleotide.

In various embodiments, compositions of the disclosure include a further aspect wherein association of the single stranded portion of the polynucleotide with the target polynucleotide causes a detectable change. Thus, in one aspect, the sicPN comprises a detectable label that causes the detectable change when the target polynucleotide is associated with the single stranded portion, while in another aspect, the target polynucleotide comprises a detectable label that causes the detectable change when the target polynucleotide is associated with the single stranded portion. In further aspects of these embodiments, the detectable label is a fluorophore, and in further aspects the fluorophore is quenched when the sicPN is associated with a polynucleotide.

In further aspects, the single stranded portion of the polynucleotide is at least about 2 nucleotides to about 100 nucleotides in length.

In one embodiment, the rate of association between the polynucleotide and the target polynucleotide is increased when a sicPN is associated with the polynucleotide compared to rate of association between the polynucleotide and the target polynucleotide in the absence of the sicPN. In various aspects, the association rate is increased by at least about 2-fold to at least about 5-fold.

With respect to the plurality of polynucleotides, the disclosure provides compositions wherein the plurality of polynucleotides are each sufficiently complementary to a target polynucleotide to allow association. In one aspect, each polynucleotide in the plurality of polynucleotides all have the same sequence. In another aspect, at least two polynucleotides in the plurality of polynucleotides have different sequences, and in yet another aspect the polynucleotides that have different sequences each have different single stranded portions that associate with different target polynucleotides.

In another embodiment, the different single stranded portions associate with the same target polynucleotide at different locations on the target polynucleotide, or the different single stranded portions associate with different target polynucleotides.

In an additional aspect, at least two sicPNs in the plurality of sicPNs have different sequences and each of the two sicPNs associate with different polynucleotides.

The disclosure also provides a method of detecting a target polynucleotide comprising contacting the target polynucleotide with a composition as described herein, wherein contact between the target and the composition results in a detectable change. In another embodiment, a method is provided for inhibiting expression of a gene product encoded by a target polynucleotide comprising contacting the target polynucleotide with a composition as described herein under condition sufficient to inhibit expression of the gene product. In various aspects, the gene product is inhibited in vivo or in vitro. In a further aspect, it is contemplated that the expression is inhibited by at least about 5%.

### DESCRIPTION OF THE DRAWINGS

FIG. 1 shows that sicDNA increases the rate of association of DNA-Au NPs to target DNA strands. (a) Scheme depicting the fluorescence-based measurement of a DNA-Au NP binding a target. (b) Progress curves of hybridization in the presence of different complements (single stranded DNA (ssDNA), short internal complement DNA (sicDNA), short external complement DNA (secDNA), long internal complement DNA (licDNA) and full complement DNA (fcDNA)). (c) Rate of DNA-Au NPs binding to targets with increasing concentrations of sicDNA present. (d) Quantification of k_{obs} from each curve in FIG. 1c plotted as a function of sicDNA/NP. (e) Plot of k_{obs} as a function of DNA-Au NP concentration. These plots were fit to linear regression curves and used to calculate kₒₙ and kₐ (f) Comparison of ssDNA and sicDNA target binding in the absence of the nanoparticle. Inset: scheme of experiment using a molecular quencher. All plots represent average values from three independent experiments. Error bars represent the standard deviation from the three independent experiments.

FIG. 2 depicts the effect of sicDNA on the bound strand and adjacent ssDNA sites. (a) Scheme of a nanoparticle containing a mixed monolayer of DNA. The different sequences can be orthogonally addressed by the corresponding sicDNA and target. This experiment was performed in the presence of both target-1 and -2, distinguished by different fluorophore labels. (b) Plot of target-1 binding to DNA-Au NPs in the presence of sicDNA-1 or -2. (b) Plot of target-2 binding to DNA-Au NPs in the presence of sicDNA-1 or -2. All plots represent average values from three independent experiments.

FIG. 3 depicts the rate of sicDNA release from DNA-Au NPs in response to target binding. (a) Scheme depicting fluorescence-based measurements of the sicDNA release. In these experiments the sicDNA, rather than the target was labeled. (b) Plot of sicDNA released as a function of target added. The dotted line represents calculated sicDNA release assuming target DNA exhibits equal binding to ssDNA and sicDNA bound sites. The relatively efficient release of sicDNA observed indicates preferential target binding to sicDNA-bound sites and selective release of sicDNA. Error bars represent the standard deviation from three experiments. For some points, the error bars are not visible because they are obscured by the mark for the data point.

FIG. 4 shows DNA conformation on the Au NP surface as a function of sicDNA concentration. (a) DLS measurements of the nanoparticle radii at different sicDNA concentrations. (b) Fluorescence spectra from DNA-Au NPs containing a distal fluorophore label. These spectra were taken before and after the addition of sicDNA. All plots represent average values from three independent experiments. Error bars represent the standard deviation from the three experiments.

FIG. 5 depicts Molecular Dynamics (MD) simulation snapshots of ssDNA and sicDNA on flat gold surfaces. Seven strands were modeled on each surface. (a) ssDNA is shown with the last nine residues above the dashed line at 10.6 nm. (b) sicDNA is shown with the last nine residues above the dashed line at 11.8 nm. (c) The normalized distribution of the distance (z) of the last residue of ssDNA (black) and sicDNA (dashed) from the surface. The average of z of ssDNA is 10.6±0.9 nm, and it is 11.8±1.0 nm for sicDNA.

FIG. 6 shows that sicDNA increases the rate of target association on microarrays. (A) Scheme depicting the fluorescence-based detection of target binding to the microarray surface. (B) Fluorescence confocal microscopy images of representative spots after exposure to the labeled target. The reaction was stopped at different time points by washing away unbound target. (C) Quantification of the fluorescence experiments shown in FIG. 6b. The initial rate of target association was determined by a linear fit of the data. Error bars represent the standard deviation from four independent experiments.

FIG. 7 depicts (A) Codelink slides functionalized with DNA in the presence or absence of complement. (B) In samples containing the displaceable duplex a stronger signal rapidly appeared.

FIG. 8 shows that displacement complements are released from a surface in response to target binding.

FIG. 9 shows DNA (black) functionalized gold nanoparticle (sphere) bind target nucleic acids (gray). This process can be monitored using fluorescently labeled target polynucleotides. When the fluorophore is bound to the nanoparticle it is quenched by the gold surface, allowing determination of association rates. When a short internal complement (sic) is bound to the nanoparticle it can be displaced and/or released by the longer target (B). The overall rate of target association increases when short complements are present.

### DETAILED DESCRIPTION OF THE INVENTION

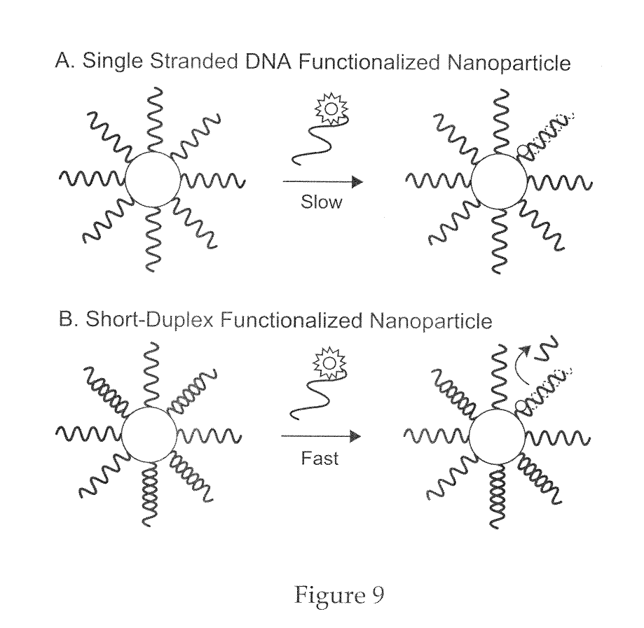

The disclosure provides compositions and methods relating to the use of short polynucleotide duplexes for enhanced association with a target polynucleotide. In this approach, a polynucleotide capable of associating with a target polynucleotide is functionalized to a surface. Additionally, a short internal complement polynucleotide (sicPN) is added that overlaps with a portion of the target polynucleotide binding site on the functionalized polynucleotide, but not the complete sequence (FIG. 9). “sicPN,” as used herein, means short internal complement polynucleotide and is understood to be a polynucleotide that associates with a polynucleotide that is functionalized to a surface, and that is displaced and/or released when a target polynucleotide hybridizes to the polynucleotide that is functionalized on the surface. In one aspect, the sicPN has a lower binding affinity or binding avidity for the functionalized polynucleotide such that association of the target molecule with the functionalized polynucleotide causes the sicPN to be displaced and/or released from its association with the functionalized polynucleotide.

Thus, there remains a single stranded portion of the functionalized polynucleotide. When the target polynucleotide associates with the single stranded portion of the functionalized polynucleotide, it displaces and/or releases the sicPN and results in an enhanced association rate of the surface-functionalized polynucleotide with the target polynucleotide.

In an embodiment, the disclosure provides compositions comprising a surface functionalized with a plurality of polynucleotides, each polynucleotide in the plurality functionalized to the surface at a terminus of the polynucleotide, the composition further comprising a plurality of short internal complementary polynucleotides (sicPNs) having a sequence sufficiently complementary to a portion of each polynucleotide in the plurality such that under appropriate conditions, a sicPN in the plurality of sicPNs is able to associate with each polynucleotide over a portion of each polynucleotide, the portion of each polynucleotide located proximal to the terminus of each polynucleotide that is functionalized to the surface, each polynucleotide having a length longer than each sicPN in the plurality to provide a single stranded portion of each polynucleotide when a polynucleotide in the plurality is associated with a sicPN in the plurality of sicPNs, the single stranded portion of the polynucleotide located distal to the portion of the polynucleotide to which the sicPN associates, the single stranded portion having a sequence sufficiently complementary to a target polynucleotide to associate with the target polynucleotide under appropriate conditions, wherein association of the polynucleotide with the target polynucleotide displaces and/or releases the sicPN associated with the polynucleotide, and wherein at least 25% of all polynucleotides in the plurality are associated with a sicPN.

In some embodiments, the plurality of sicPNs are each the same length. In further embodiments, the plurality of sicPNs are of different lengths, as long as the single stranded portion of the functionalized polynucleotide that is not associated with a sicPN is capable of associating with the target polynucleotide and each sicPN is displaced and/or released upon the association. In various aspects, the single stranded portion of the functionalized polynucleotide is at least about 2 nucleotides to about 100 nucleotides in length. In further aspects, the single stranded portion of the functionalized polynucleotide is at least about 5 to about 75 nucleotides, about 10 to about 50 nucleotides, about 15 to about 40 nucleotides, or about 20 to about 30 nucleotides in length. Accordingly, the single stranded portion of the functionalized polynucleotide is, in various aspects, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length.

In various aspects, a surface is functionalized with a plurality of polynucleotides, wherein the polynucleotides in the plurality can be directed against one or more than one target polynucleotide. Accordingly, a plurality of sicPNs can be added that can associate individually with one or more polynucleotides functionalized on the surface. In some aspects, this will preferentially enhance the rate of hybridization of a specific group of polynucleotides functionalized on the surface, relative to the hybridization of a second group of functionalized polynucleotides that have a different sequence. One of ordinary skill will understand that various combinations of polynucleotides can be functionalized to a surface, and these polynucleotides can be designed to associate with one or more target polynucleotides. Further, one or more sicPNs can be designed and implemented to enhance or retard the rate of association with any one or multiple target polynucleotides. In additional aspects, use of more than one surface, functionalized with a plurality of polynucleotides each comprising the same or different sequences, is contemplated for association with a plurality of sicPNs.

It is disclosed herein that the rate of target polynucleotide hybridization to a polynucleotide functionalized on a surface is related to the number of functionalized polynucleotides that are in association with a sicPN. In general, a higher percentage of functionalized polynucleotides that are in association with a sicPN relates to a higher rate of hybridization between the functionalized polynucleotide and the target polynucleotide. Thus, in one aspect, at least about 25% of a plurality of polynucleotides that are functionalized on a surface are in association with a sicPN. In another aspect, from at least about 15% to at least about 75% of a plurality of polynucleotides that are functionalized on a surface are in association with a sicPN. In a further aspect, from at least about 25% to at least about 50%, or from at least about 40% to at least about 80%, or from at least about 50% to at least about 95% of a plurality of polynucleotides that are functionalized on a surface are in association with a sicPN. In various aspects, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more of a plurality of polynucleotides that are functionalized on a surface are in association with a sicPN.

As described above, when the target polynucleotide associates with the single stranded portion of the functionalized polynucleotide, it displaces and/or releases the sicPN and results in an enhanced association rate of the surface-functionalized polynucleotide with the target polynucleotide compared to an association rate in the absence of the sicPN. In general, any increase in the association rate is contemplated. In an aspect, the association rate is increased by at least about 2-fold to at least about 100-fold or more. In further aspects, the association rate is increased by at least about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, about 50-fold, about 55-fold, about 60-fold, about 65-fold, about 70-fold, about 75-fold, about 80-fold, about 85-fold, about 90-fold, about 95-fold, about 100-fold or more. In still further aspects, the association rate is increased by at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50% or more compared to an association rate in the absence of the sicPN.

The sicPN provides opportunities to build additional functionality into the methods provided. First, the sicPN binds in the recognition region of a surface-functionalized polynucleotide, directly affecting secondary structure of the functionalized polynucleotide that is functionalized to the surface. Since secondary structure is a major source of false-negatives in surface assays, in one aspect, a sicPN is used to rescue the activity of the polynucleotide probe, improving the reproducibility of any assay involving polynucleotide hybridization at surfaces.

“Proximal” and “distal” when used in reference to a polynucleotide refer to a location on the polynucleotide and is measured in nucleotides. The terms are understood in one aspect to use as a reference point a surface to which the polynucleotide is functionalized. A nucleotide or portion of a polynucleotide that is said to be “proximal” to another nucleotide or portion of a polynucleotide is understood to be closer, by one or more nucleotides, to the surface. Likewise, a nucleotide or portion of a polynucleotide that is said to be “distal” to another nucleotide or portion of a polynucleotide is understood to be farther, by one or more nucleotides, from the surface.

“Displace” as used herein means that a sicPN is partially denatured from its association with a polynucleotide. A displaced sicPN is still in partial association with the polynucleotide to which it is associated. “Release” as used herein means that the sicPN is sufficiently displaced (i.e., completely denatured) so as to cause its disassociation from the polynucleotide to which it is associated. In some aspects wherein the sicPN comprises a detectable marker, it is contemplated that the release of the sicPN causes the detectable marker to be detected.

It is noted here that, as used in this specification and the appended claims, the singular forms “a,” “an,” and “the” include plural reference unless the context clearly dictates otherwise.

It is further noted that the terms “attached,” “conjugated” and “functionalized” are also used interchangeably herein and refer to the association of a polypeptide, a polynucleotide or combinations of a polypeptide and polynucleotide with a nanoparticle.

It is also noted that the term “about” as used herein is understood to mean approximately.

“Hybridization” means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

A “complex” as used herein comprises a target molecule in association with a surface. A complex arises from hybridization of a target polynucleotide with a polynucleotide functionalized on a surface or interaction between a target polypeptide with an aptamer functionalized on a surface.

### Polynucleotides

Polynucleotides contemplated by the present disclosure include DNA, RNA, modified forms and combinations thereof as defined herein. A polynucleotide as disclosed herein is, in some aspects, functionalized on a surface or associates with a polynucleotide that is functionalized on a surface. In these aspects, the polynucleotide recognizes and associates with a target polynucleotide as defined herein. Accordingly, in some aspects, a polynucleotide is a molecule that is recognized by and associates with a functionalized surface.

A “polynucleotide” is understood in the art to comprise individually polymerized nucleotide subunits. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N-6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleotides include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Surfaces provided that are functionalized with a polynucleotide, or a modified form thereof, generally comprise a polynucleotide from about 5 nucleotides to about 100 nucleotides in length. More specifically, nanoparticles are functionalized with polynucleotides that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length are contemplated.

Polynucleotides, as defined herein, also includes aptamers. The production and use of aptamers is known to those of ordinary skill in the art. In general, aptamers are nucleic acid or peptide binding species capable of tightly binding to and discreetly distinguishing target ligands [Yan et al., RNA Biol. 6(3) 316-320 (2009), incorporated by reference herein in its entirety]. Aptamers, in some embodiments, may be obtained by a technique called the systematic evolution of ligands by exponential enrichment (SELEX) process [Tuerk et al., Science 249:505-10 (1990), U.S. Pat. No. 5,270,163, and U.S. Pat. No. 5,637,459, each of which is incorporated herein by reference in their entirety]. General discussions of nucleic acid aptamers are found in, for example and without limitation, Nucleic Acid and Peptide Aptamers: Methods and Protocols (Edited by Mayer, Humana Press, 2009) and Crawford et al., Briefings in Functional Genomics and Proteomics 2(1): 72-79 (2003). In various aspects, an aptamer is between 10-100 nucleotides in length.

### Modified Polynucleotides

As discussed above, modified polynucleotides are contemplated for functionalizing surfaces. In various aspects, a polynucleotide functionalized on a surface is completely modified or partially modified. Thus, in various aspects, one or more, or all, sugar and/or one or more or all internucleotide linkages of the nucleotide units in the polynucleotide are replaced with “non-naturally occurring” groups.

In one aspect, this embodiment contemplates a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotide.”

Modified polynucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the polynucleotide consists of 2 to 4, desirably 3, groups/atoms selected from —CH₂—, —O—, —S—, —NRH—, >C═O, >C═NRH, >C═S, —Si(R″)₂—, —SO—, —S(O)₂—, —P(O)₂—, —PO(BH₃) —, —P(O,S)—, —P(S)₂—, —PO(R″)—, —PO(OCH₃)—, and —PO(NHRH)—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH2-O—, —O—CH2-CH2-, —O—CH2-CH=(including R5 when used as a linkage to a succeeding monomer), —CH₂—CH₂—O—, —NRH—CH₂—CH₂—, —CH₂—CH₂—NRH—, —CH₂—NRH—CH₂—-, —O—CH₂—CH₂—NRH—, —NRH—CO—O—, —NRH—CO—NRH—, —NRH—CS—NRH—, —NRH—C(═NRH)—NRH—, —NRH—CO—CH₂—NRH—O—CO—O—, —O—CO—CH₂—, —O—CH₂—CO—O—, —CH₂—CO—NRH—, —O—CO—NRH—, —NRH—CO—CH₂—, —CH₂—CO—NRH —, —O—CH₂—CH₂—NRH—, —CH═N—O—, —CH₂—NRH—O—, —CH₂—O—N=(including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, —S—CH₂—O—, —CH₂—CH₂—S—, —O—CH₂—CH₂—S—, —S—CH₂CH=(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O, —O—S(O)₂—O—, —O—S(O)₂—CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHRN)—O—, —O—P(O)₂—NRH H—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—O—P(—O,S)—O—, —O—P(S)₂—O—, —NRH P(O)₂—O—, —O—P(O,NRH)—O—, —O—PO(R″)—O—, —O—PO(CH₃)—O—, and —O PO(NHRN)—O—, where RH is selected form hydrogen and C₁₋₄-alkyl, and R″ is selected from C₁₋₆-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Application No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C_{I} to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)ₙ group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Polynucleotide Attachment to a Surface

Polynucleotides contemplated for use in the methods include those bound to a surface through any means. Regardless of the means by which the polynucleotide is attached to the surface, attachment in various aspects is effected through a 5′ linkage, a 3′ linkage, some type of internal linkage, or any combination of these attachments.

In some embodiments, the polynucleotide attached to a surface is DNA. When DNA is attached to a surface, the DNA is, in some aspects, comprised of a sequence that is sufficiently complementary to a target sequence of a polynucleotide such that hybridization of the DNA polynucleotide attached to a surface and the target polynucleotide takes place, thereby associating the target polynucleotide with the surface and causing displacement and/or release of a sicPN as described herein. The DNA in various aspects is single stranded or double stranded, as long as the double stranded molecule also includes a single strand sequence that hybridizes to a single strand sequence of the target polynucleotide. In some aspects, hybridization of the polynucleotide functionalized on the surface can form a triplex structure with a double-stranded target polynucleotide. In another aspect, a triplex structure can be formed by hybridization of a double-stranded polynucleotide functionalized on a nanoparticle to a single-stranded target polynucleotide.

In some embodiments, the disclosure contemplates that a polynucleotide attached to a surface is RNA. When RNA is attached to a surface, the RNA is, in some aspects, comprised of a sequence that is sufficiently complementary to a target sequence of a polynucleotide such that hybridization of the RNA polynucleotide attached to a surface and the target polynucleotide takes place, thereby associating the target polynucleotide with the surface and causing displacement and/or release of a sicPN as described herein. The RNA in various aspects is single stranded or double stranded, as long as the double stranded molecule also includes a single strand sequence that hybridizes to a single strand sequence of the target polynucleotide. In some aspects the RNA attached to a surface is a small interfering RNA (siRNA).

The surfaces contemplated by the disclosure include without limitation a nanoparticle and a solid support. Thus, in some aspects, the surface functionalized with a polynucleotide is a nanoparticle. Methods of polynucleotide attachment to a nanoparticle are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Accordingly, in some embodiments, the disclosure contemplates that a polynucleotide attached to a nanoparticle is RNA. In a further embodiment, a polynucleotide attached to a nanoparticle is

Nanoparticles as provided herein have a packing density of the polynucleotides on the surface of the nanoparticle that is, in various aspects, sufficient to result in cooperative behavior between nanoparticles and between polynucleotide strands on a single nanoparticle. In another aspect, the cooperative behavior between the nanoparticles increases the resistance of the polynucleotide to nuclease degradation. In yet another aspect, the uptake of nanoparticles by a cell is influenced by the density of polynucleotides associated with the nanoparticle. As described in PCT/US2008/65366, incorporated herein by reference in its entirety, a higher density of polynucleotides on the surface of a nanoparticle is associated with an increased uptake of nanoparticles by a cell.

A surface density adequate to make the nanoparticles stable and the conditions necessary to obtain it for a desired combination of nanoparticles and polynucleotides can be determined empirically. Generally, a surface density of at least 2 pmoles/cm² will be adequate to provide stable nanoparticle-polynucleotide compositions. In some aspects, the surface density is at least 15 pmoles/cm². Methods are also provided wherein the polynucleotide is bound to the nanoparticle at a surface density of from at least about 2 μmol/cm² to at least about 1000 μmol/cm² or more.

In some embodiments, the polynucleotide is covalently or non-covalently coupled to a solid support. Coupling chemistries and selection of support materials well known in the art are contemplated. A non-limiting example of the attachment of a polynucleotide to a solid support is provided herein (see Example 6) [See also Lipshutz et al., Nanotechnology 14 (7): R15-R27 (2003), and U.S. Pat. Nos. 5,252,743; 5,412,087; 5,445,934; 5,658,802; 5,700,637; 5,774,305; 6,054,270, each of which is incorporated herein by reference in their entirety]. The optimal density of polynucleotides on a solid support can be determined empirically, and is within the ordinary skill in the art.

### Methods of Labeling Polynucleotides

A polynucleotide as described herein, in various aspects, further comprises a detectable label. Accordingly, the disclosure provides compositions and methods wherein polynucleotide complex formation is detected by a detectable change. In one aspect, complex formation gives rise to a color change which is observed with the naked eye or spectroscopically.

Methods for visualizing the detectable change resulting from polynucleotide complex formation include any fluorescent detection method, including without limitation fluorescence microscopy, a microtiter plate reader or fluorescence-activated cell sorting (FACS).

It will be understood that a label contemplated by the disclosure includes any of the fluorophores described herein as well as other detectable labels known in the art. For example, labels also include, but are not limited to, redox active probes, chemiluminescent molecules, radioactive labels, dyes, fluorescent molecules, phosphorescent molecules, imaging agents including but not limited to gadolinium, quantum dots, as well as any marker which can be detected using spectroscopic means, i.e., those markers detectable using microscopy and cytometry. In aspects of the disclosure wherein a detectable label is to be detected, the disclosure provides that any luminescent, fluorescent, or phosphorescent molecule or particle can be efficiently quenched by noble metal surfaces. Accordingly, each type of molecule is contemplated for use in the compositions and methods disclosed.

Methods of labeling polynucleotides with fluorescent molecules and measuring fluorescence are well known in the art. Suitable fluorescent molecules are also well known in the art and include without limitation 1,8-ANS (1-Anilinonaphthalene-8-sulfonic acid), 1-Anilinonaphthalene-8-sulfonic acid (1,8-ANS),5-(and-6)-Carboxy-2′,7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, high Ca, Fura-2, no Ca, GFP(S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Niss1 stain-RNA, Nile Blue, EtOH, Nile Red, Nile Red-lipid, Niss1, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PicoGreen dsDNA quantitation reagent, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, EtOH, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

### Surfaces

### Nanoparticles

In some embodiments, nanoparticles are provided which are functionalized to have a polynucleotide attached thereto. The size, shape and chemical composition of the nanoparticles contribute to the properties of the resulting polynucleotide-functionalized nanoparticle. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nanometer (nm) to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

### Solid Supports

Numerous assays rely on this type of surface and include but are not limited to microarrays, chip-based assays, ELISA-style assays and the bio-barcode. The use of sicPNs increases the rate of target-surface association and thus decreases the total time required to run these assays. Additionally, a major problem in highly multiplexed techniques (for example and without limitation, microarrays) is the formation of secondary structure in surface-functionalized polynucleotides. The use of sicPNs can rescue the activity of these probes, resulting in more uniform signal from different spots on the microarray and fewer false negatives.

Accordingly, in some embodiments a polynucleotide is functionalized to a solid support. For example and without limitation, supports include those made all or in part of glass, silica, metal, plastic, fiber, resin, and polymers. Exemplary polymers include for example and without limitation cellulose, nitrocellulose, polyacetate, polycarbonate, polystyrene, polyester, polyvinyldifluorobenzene, nylon, carbon fiber or any other suitable polymer material. In certain related embodiments one or a plurality of the polynucleotides described herein may be provided as an array immobilized on a solid support, which includes any of a number of well known configurations for spatially arranging such molecules in an identifiable (for example and without limitation, addressable) fashion. The skilled artisan will be familiar with various compositions and methods for making and using arrays of such solid-phase immobilized polynucleotide arrays.

### Target Polynucleotides

In some embodiments, the present disclosure is directed to contacting a target polynucleotide with a functionalized surface to form a complex, and further comprising displacing and/or releasing a sicPN to enable the detection of the target polynucleotide.

In various aspects, the target polynucleotide is either eukaryotic, prokaryotic, or viral.

For prokaryotic target polynucleotides, in various aspects, the polynucleotide is genomic DNA or RNA transcribed from genomic DNA. For eukaryotic target polynucleotides, the polynucleotide is an animal polynucleotide, a plant polynucleotide, a fungal polynucleotide, including yeast polynucleotides. As above, the target polynucleotide is either a genomic DNA or RNA transcribed from a genomic DNA sequence. In certain aspects, the target polynucleotide is a mitochondrial polynucleotide. For viral target polynucleotides, the polynucleotide is viral genomic RNA, viral genomic DNA, or RNA transcribed from viral genomic DNA.

In various embodiments, methods provided include those wherein the target polynucleotide is a mRNA encoding a gene product and translation of the gene product is inhibited, or the target polynucleotide is DNA in a gene encoding a gene product and transcription of the gene product is inhibited. In methods wherein the target polynucleotide is DNA, the polynucleotide is in certain aspects DNA which encodes the gene product being inhibited. In other methods, the DNA is complementary to a coding region for the gene product. In still other aspects, the DNA encodes a regulatory element necessary for expression of the gene product. “Regulatory elements” include, but are not limited to enhancers, promoters, silencers, polyadenylation signals, regulatory protein binding elements, regulatory introns, ribosome entry sites, and the like. In still another aspect, the target polynucleotide is a sequence which is required for endogenous replication.

The terms “start codon region” and “translation initiation codon region” refer to a portion of an mRNA or gene that encompasses contiguous nucleotides in either direction (i.e., 5′ or 3′) from a translation initiation codon. Similarly, the terms “stop codon region” and “translation termination codon region” refer to a portion of such an mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the polynucleotides on the functionalized surfaces.

Other target regions include the 5′ untranslated region (5′UTR), the portion of an mRNA in the 5′ direction from the translation initiation codon, including nucleotides between the 5′ cap site and the translation initiation codon of an mRNA (or corresponding nucleotides on the gene), and the 3′ untranslated region (3′UTR), the portion of an mRNA in the 3′ direction from the translation termination codon, including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site.

Each surface utilized in the methods provided has a plurality of polynucleotides attached to it. As a result, each surface-polynucleotide conjugate has the ability to bind to a plurality of target polynucleotides having a sufficiently complementary sequence. For example and without limitation, if a specific mRNA is targeted, a single surface has the ability to bind to multiple copies of the same transcript. In one aspect, methods are provided wherein the surface is functionalized with identical polynucleotides, i.e., each polynucleotide has the same length and the same sequence. In other aspects, the surface is functionalized with two or more polynucleotides which are not identical, i.e., at least one of the attached polynucleotides differ from at least one other attached polynucleotide in that it has a different length and/or a different sequence. In aspects wherein different polynucleotides are attached to the surface, these different polynucleotides bind to the same single target polynucleotide but at different locations, or bind to different target polynucleotides which encode different gene products. Accordingly, in various aspects, a single functionalized surface may be used in a method to inhibit expression of more than one gene product. Polynucleotides are thus used to target specific polynucleotides, whether at one or more specific regions in the target polynucleotide, or over the entire length of the target polynucleotide as the need may be to effect detection of the target polynucleotide, or a desired level of inhibition of gene expression. Accordingly, the polynucleotides are designed with knowledge of the target sequence. Methods of making polynucleotides of a predetermined sequence have been described herein.

In various aspects, target polynucleotides contemplated by the present disclosure include but are not limited to genomic DNA and/or mRNA encoding cancer antigen 150 (CA150), Cancer antigen (CA19), cancer antigen (CA50), calcium binding protein 39-like (CAB39L), CD22, CD24, CD5, CD19, CD63, CD66, Carcinoembryonic antigen-related cell adhesion molecule 1 (biliary glycoprotein) (CEACAM1), carcinoembryonic antigen-related cell adhesion molecule 5 (CEACAM5), clusterin associated protein 1 (CLUAP1), cancer/testis antigen 1B (CTAG1B), cancer/testis antigen 2 (CTAG2), cutaneous T-cell lymphoma-associated antigen 5 (CTAGE5), carcinoembryonic antigen (CEA), estrogen receptor-binding fragment-associated antigen 9 (EBAG9), FAM120C, FLJ14868, formin-like protein 1 (FMNL1), G antigen 1 (GAGE1), glycoprotein A33 (transmembrane) (GPA33), ganglioside OAcGD3, heparanase 1, Jak and microtubule interacting protein 2 (JAKMIP2), leucine-rich repeats and immunoglobulin-like domains 3 (LRIG3), leucine rich repeat containing 15 (LRRC15), lung carcinoma Cluster 2, melanoma-associated antigen 1 (MAGE 1), melanoma antigen family A, 10 (MAGEA10), melanoma antigen family A, 11 (MAGEA11), melanoma antigen family A, 12 (MAGEA12), melanoma antigen family A, 2 (MAGEA2), melanoma antigen family A, 4 (MAGEA4), melanoma antigen family B, 1 (MAGEB1), melanoma antigen family B, 2 (MAGEB2), melanoma antigen family B, 3 (MAGEB3), melanoma antigen family B, 4 (MAGEB4), melanoma antigen family B, 6 (MAGEB6), melanoma antigen family C, 1 (MAGEC1), melanoma antigen family E, 1 (MAGEE1), melanoma antigen family H, 1 (MAGEH1), melanoma antigen family L 2 (MAGEL2), meningioma expressed antigen 5 (hyaluronidase), (MGEA5), MOK protein kinase, mucin 16, cell surface associated (MUC16), mucin 4, cell surface associated (MUC4), melanoma associated antigen, mesothelin, mucin 5AC, nestin, ovarian cancer immuno-reactive antigen domain containing 1 (OCIAD1), opa interacting protein 5 (OIP5), ovarian carcinoma-associated antigen, PAGE4, proliferating cell nuclear antigen (PCNA), preferentially expressed antigen in melanoma (PRAME), prostate tumor overexpressed 1 (PTOV1), plastin L, prostate cell surface antigen, prostate mucin antigen/PMA, RAGE, RASD2, ring finger protein 43 (RNF43), ropporin, rhophilin associated protein 1 (ROPN1), ribosomal protein, large, P2 (RPLP2), squamous cell carcinoma antigen recognized by T cell 2 (SART2), squamous cell carcinoma antigen recognized by T cells 3 (SART3), small breast epithelial mucin (SBEM), serologically defined colon cancer antigen 10 (SDCCAG10), serologically defined colon cancer antigen 8 (SDCCAG8), sel-1 suppressor of 11n-12-like (C. elegans) (SEL1L), human sperm protein associated with the nucleus on the X chromosome (SPANX), SPANXB1, synovial sarcoma, X breakpoint 5 (SSX5), six-transmembrane epithelial antigen of prostate 4 (STEAP4), serine/threonine kinase 31 (STK31), tumor associated glycoprotein (TAG72), tumor endothelial marker 1 (TEM1), X antigen family, member 2 (XAGE2). Additional target polynucleotides contemplated by the present disclosure include without limitation genomic DNA and/or mRNA encoding cardiac markers (for example and without limitation, troponin) and/or viral markers (for example and without limitation, HIV p24).

Of course, the skilled artisan can easily design a polynucleotide sequence that associates with any desired target polynucleotide. The present disclosure is therefore not limited in scope by the target molecules disclosed herein.

### Methods

### General

Methods provided by the disclosure center on increasing the rate at which a polynucleotide associates with a particular target polynucleotide. Aspects of the general method include detecting the target polynucleotide, and/or inhibiting the expression of the target polynucleotide. As described herein, the increased rate of association between the polynucleotide and the target polynucleotide is achieved through the use of a sicPN, which associates with a portion of the polynucleotide, the increase in rate is compared to the same association reaction in the absence of the sicPN. The duplex that is formed by the association of the functionalized polynucleotide and the sicPN is such that a single stranded portion of the functionalized polynucleotide is then available to recognize and associate with the target polynucleotide.

The association of the polynucleotide with the target polynucleotide additionally displaces and, in some aspects, releases the sicPN. The sicPN or the target polynucleotide, in various embodiments, further comprise a detectable label. Thus, in one aspect of a method wherein detection of the target polynucleotide is desired, it is the displacement and/or release of the sicPN that generates the detectable change through the action of the detectable label. In another method wherein detection of the target polynucleotide is desired, it is the target polynucleotide that generates the detectable change through its own detectable label. In methods wherein inhibition of the target polynucleotide expression is desired, it is the association of the functionalized polynucleotide with the target polynucleotide that generates the inhibition of target polynucleotide expression through an antisense mechanism.

The compositions of the disclosure comprise a plurality of sicPNs, able to associate with a plurality of polynucleotides, that may be used on one or more surfaces to specifically associate with a plurality of target polynucleotides. Thus, the steps or combination of steps of the methods described below apply to one or a plurality of functionalized polynucleotides, sicPNs and target polynucleotides.

In various aspects, the methods include use of a polynucleotide which is 100% complementary to the target polynucleotide, i.e., a perfect match, while in other aspects, the polynucleotide is at least (meaning greater than or equal to) about 95% complementary to the polynucleotide over the length of the polynucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the polynucleotide to the extent that the polynucleotide is able to achieve the desired degree of inhibition of a target gene product. It will be understood by those of skill in the art that the degree of hybridization is less significant than a resulting detection of the target polynucleotide, or a degree of inhibition of gene product expression.

The method can thus be generalized as including the following steps depending on the desired application. A first hybridization between the functionalized polynucleotide and the sicPN, and a second hybridization step between the functionalized polynucleotide and the target polynucleotide, which results in displacement and/or release of the sicPN from the functionalized polynucleotide. Additional steps that may be performed include a surface functionalization step, a labeling step, and a detection step. To carry out the methods provided, a surface is optionally functionalized with a polynucleotide (surface functionalization step), to which a sicPN is hybridized (first hybridization step). The sicPN is optionally labeled with a detectable label (labeling step). The functionalized surface is contacted with a target polynucleotide (second hybridization step) and is incubated under conditions sufficient to allow the hybridization of the target polynucleotide to the functionalized polynucleotide (second hybridization step). A washing step optionally follows the hybridization step. The hybridization results in the displacement and/or release of the sicPN (displacement and/or release step), and in aspects wherein the sicPN has been labeled with a detectable label, the displacement and/or release allows for the detection of the sicPN (detection step), detection indicating the presence of the target polynucleotide. In other aspects, the target polynucleotide is labeled with a detectable label (labeling step) which indicates the presence of the target polynucleotide when it is hybridized to the functionalized polynucleotide.

As mentioned above, the steps that are performed will depend on the particular application. By way of example, the compositions of the disclosure have the property of increasing the rate of hybridization of a polynucleotide with a target polynucleotide against which it is directed. Thus, in some aspects the use of a detectable label is not required. As such, in some aspects a detection step is also not required.

It is also contemplated that in some aspects, a surface functionalization step is not required. By way of example, a polynucleotide that is associated with a sicPN can be used directly in an assay to inhibit gene expression, without need for a surface, as long as at least about 25% of the population of polynucleotides is in association with a sicPN. It is further contemplated that the washing step is optional, for example and without limitation in aspects wherein the composition is used intracellularly to detect or inhibit a target polynucleotide.

The individual steps used in various methods are described in more detail below.

Surface Functionalization Step. A surface as described herein includes a nanoparticle and a solid support. Any surface to which a polynucleotide can be attached is contemplated for use, and methods for attaching a polynucleotide to a surface have been described herein.

First Hybridization Step. The first hybridization step is the association of the polynucleotide to be functionalized on a surface with a sicPN. This step can take place either before or after the polynucleotide is functionalized to a surface. In some aspects, the sicPN is labeled with a detectable label.

Hybridization conditions can be determined by those of skill in the art, and are sensitive to parameters including but not limited to temperature, ionic strength of the hybridization solution, time, pH, and degree of complementarity between the polynucleotides being hybridized. See, e.g., Sambrook et al., Molecular Cloning: A Laboratory Manual (2nd ed. 1989) for general discussion on hybridization of polynucleotides. The amount of the sicPN to be used will depend on the number of polynucleotides that are functionalized on a surface. As described herein, an amount of the sicPN is added such that at least about 25% of the functionalized polynucleotides are associated with a sicPN.

Labeling Step. The labeling step comprises the addition of a detectable label to a sicPN, a target polynucleotide, or both. Methods for attaching a detectable label to a polynucleotide are described herein, and are additionally known to those of skill in the art. In one aspect, the sicPN is labeled with a detectable marker prior to its hybridization to the polynucleotide to be functionalized on a surface. In another aspect, the sicPN is labeled with a detectable marker after its hybridization to the polynucleotide to be functionalized on a surface. In any of the aspects wherein the sicPN is labeled with a detectable label, it is contemplated that in one aspect the target polynucleotide is not labeled with a detectable label. In another aspect, the sicPN and the target polynucleotide are both labeled with a detectable label, and in an embodiment the detectable labels are distinguishable from each other. In some aspects wherein the sicPN is labeled with a detectable label, it is contemplated that the detectable label is quenched due to its proximity to a surface.

In another aspect, the target polynucleotide is labeled with a detectable label and the sicPN is not labeled with a detectable label.

Second Hybridization Step. The second hybridization step is the association of the functionalized polynucleotide with the target polynucleotide. Hybridization conditions have been described herein. This hybridization step can occur either in vivo or in vitro depending on the particular application. This hybridization can be incubated for minutes to hours or more to allow the association of the functionalized polynucleotide with the target polynucleotide. In general, the incubation is allowed to proceed for 30 minutes at room temperature but can be incubated for about 30 seconds to about 72 hours or more at between about 4° C. and about 95° C. The time and temperature of the incubation will depend on the particular application, and can be determined by one of skill in the art without undue experimentation.

Displacement and/or Release Step. It has been disclosed herein that hybridization of a functionalized polynucleotide with a target polynucleotide causes displacement and/or release of the sicPN from the functionalized polynucleotide. The displacement and/or release results from the invasion of the target polynucleotide onto the functionalized polynucleotide. The sicPN is either displaced (i.e., partially denatured) or released (i.e., completely denatured) as a result of the hybridization of a functionalized polynucleotide with a target polynucleotide.

Washing Step. In aspects wherein a target polynucleotide comprises a detectable label, a washing step optionally follows the hybridization step, wherein unbound or non specifically hybridized target polynucleotides are removed from the assay to eliminate false positive results. Accordingly, any of the methods of the disclosure may be carried out without a washing step.

Washing conditions are known to those of skill in the art and can be determined empirically, but generally involve successive rounds of adding and removing a buffer solution and then assessing the resulting assay for a detectable change. The buffer solution typically comprises salt, a detergent or both, and can increase in stringency through the successive rounds of washing. A higher stringency (i.e., a condition requiring a tighter association of a polynucleotide with a target polynucleotide in order to stay associated with each other) is generally achieved by decreasing a salt concentration and/or a detergent concentration.

Detection Step. Displacement and/or release of a sicPN that in one aspect comprises a detectable label indicates the hybridization between the functionalized polynucleotide and the target polynucleotide. The ability to detect the detectable label depends on the increase in distance between the detectable label present on the sicPN and the quenching surface. In some aspects, a labeled sicPN that is displaced is far enough away from the quenching surface to be detected. In other aspects, a sicPN must be released in order to be detected.

### Methods of Detecting a Target Polynucleotide

The disclosure provides methods of detecting a target polynucleotide comprising contacting the target polynucleotide with a composition as described herein, the contacting resulting in a detectable change, wherein the detectable change indicates the detection of the target polynucleotide. Detection of the detectable label is performed by any of the methods described herein.

As described herein, it is the displacement and/or release of the sicPN that generates the detectable change. The detectable change is assessed through the use of a detectable label, and in one aspect, the sicPN is labeled with the detectable label. Further according the methods, the detectable label is quenched when in proximity with a surface to which it is functionalized. While it is understood in the art that the term “quench” or “quenching” is often associated with fluorescent markers, it is contemplated herein that the signal of any marker that is quenched when it is relatively undetectable. Thus, it is to be understood that methods exemplified throughout this description that employ fluorescent markers are provided only as single embodiments of the methods contemplated, and that any marker which can be quenched can be substituted for the exemplary fluorescent marker.

The sicPN is thus associated with the functionalized polynucleotide in such a way that the detectable label is in proximity to the surface to quench its detection. When the functionalized polynucleotide comes in contact and associates with the target polynucleotide, it causes displacement and/or release of the sicPN. The release of the sicPN thus increases the distance between the detectable label present on the sicPN and the surface to which the polynucleotide is functionalized. This increase in distance allows detection of the previously quenched detectable label, and indicates the presence of the target polynucleotide.

Thus, in one embodiment a method is provided in which a plurality of polynucleotides are functionalized to a surface by a method known in the art and described herein. The polynucleotides are designed to be able to hybridize to one or more target polynucleotides under stringent conditions. Hybridization can be performed under different stringency conditions known in the art and as discussed herein. Under appropriate stringency conditions, hybridization between the functionalized polynucleotide and the target polynucleotide could reach about 60% or above, about 70% or above, about 80% or above, about 90% or above, about 95% or above, about 96% or above, about 97% or above, about 98% or above, or about 99% or above in the reactions. Following functionalization of a surface with the plurality of polynucleotides, a plurality of sicPNs optionally comprising a detectable label is added and allowed to hybridize with the functionalized polynucleotides. In some aspects, the plurality of polynucleotides and the sicPNs are first hybridized to each other, and then duplexes are functionalized to the surface. Regardless of the order in which the plurality of polynucleotide are hybridized to the plurality of sicPNs and the duplex is functionalized to the surface, the next step is to contact the functionalized surface with a target polynucleotide. The target polynucleotide can, in various aspects, be in a solution, or it can be inside a cell. It will be understood that in some aspects, the solution is being tested for the presence or absence of the target polynucleotide while in other aspects, the solution is being tested for the relative amount of the target polynucleotide.

After contacting the duplex with the target polynucleotide, the target polynucleotide will displace and/or release the sicPN as a result of its hybridization with the functionalized polynucleotide. The displacement and release of the sicPN allows an increase in distance between the surface and the sicPN, thus resulting in the label on the sicPN being rendered detectable. The amount of label that is detected as a result of displacement and release of the sicPN is related to the amount of the target polynucleotide present in the solution. In general, an increase in the amount of detectable label correlates with an increase in the number of target polynucleotides in the solution.

In some embodiments it is desirable to detect more than one target polynucleotide in a solution. In these embodiments, more than one sicPN is used, and each sicPN comprises a unique detectable label. Accordingly, each target polynucleotide, as well as its relative amount, is individually detectable based on the detection of each unique detectable label.

In some embodiments, the compositions of the disclosure are useful in nano-flare technology. The nano-flare is an existing class of polynucleotide functionalized nanoparticles (PN-NPs) that can take advantage of a sicPN architecture for fluorescent detection of mRNA levels inside a living cell [described in WO 2008/098248, incorporated by reference herein in its entirety]. In this system the sicPN acts as the “flare” and is detectably labeled and displaced or released from the surface by an incoming target polynucleotide.

### Methods of Inhibiting Gene Expression

Additional methods provided by the disclosure include methods of inhibiting expression of a gene product expressed from a target polynucleotide comprising contacting the target polynucleotide with a composition as described herein, wherein the contacting is sufficient to inhibit expression of the gene product. Inhibition of the gene product results from the hybridization of a target polynucleotide with a composition of the disclosure.

It is understood in the art that the sequence of a functionalized polynucleotide need not be 100% complementary to that of its target polynucleotide in order to specifically hybridize to the target polunucleotide. Moreover, a functionalized polynucleotide may hybridize to a target polynucleotide over one or more segments such that intervening or adjacent segments are not involved in the hybridization event (for example and without limitation, a loop structure or hairpin structure). The percent complementarity is determined over the length of the functionalized polynucleotide. For example, given a functionalized polynucleotide in which 18 of 20 nucleotides of the functionalized polynucleotide are complementary to a 20 nucleotide region in a target polynucleotide of 100 nucleotides total length, the functionalized polynucleotide would be 90 percent complementary. In this example, the remaining noncomplementary nucleotides may be clustered or interspersed with complementary nucleotides and need not be contiguous to each other or to complementary nucleotides. Percent complementarity of a functionalized polynucleotide with a region of a target polynucleotide can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of a polynucleotide-functionalized surface. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in vitro in a cell culture assay, generally as a predictable measure of a degree of inhibition that can be expected in vivo resulting from use of a composition as described herein. It is contemplated by the disclosure that the inhibition of a target polynucleotide is used to assess the effects of the inhibition on a given cell. By way of non-limiting examples, one can study the effect of the inhibition of a gene product wherein the gene product is part of a signal transduction pathway. Alternatively, one can study the inhibition of a gene product wherein the gene product is hypothesized to be involved in an apoptotic pathway.

It will be understood that any of the methods described herein can be used in combination to achieve a desired result. For example and without limitation, methods described herein can be combined to allow one to both detect a target polynucleotide as well as regulate its expression. In some embodiments, this combination can be used to quantitate the inhibition of target polynucleotide expression over time either in vitro or in vivo. This is achieved, in one aspect, by removing cells from a culture at specified time points and assessing the relative level of expression of a target polynucleotide at each time point. A decrease in the amount of detectable label over time indicates the rate of inhibition of the target polynucleotide. The decrease is measured using a detection method as described above.

Thus, determining the effectiveness of a given polynucleotide to hybridize to and inhibit the expression of a target polynucleotide, as well as determining the effect of inhibition of a given polynucleotide on a cell, each determined via methods described herein, are aspects that are contemplated.

### Kits

Also provided are kits for detecting a target polynucleotide and/or inhibiting gene expression from a target polynucleotide. In one embodiment, the kit comprises at least one container, the container holding at least one types of nanoparticle as described herein having one or more polynucleotides as described herein functionalized thereto. The polynucleotides on a first type of surface have one or more sequences complementary (or sufficiently complementary to permit hybridization) to one or more sequences of a target polynucleotide. The container also includes one or more sicPNs that are complementary or sufficiently complementary to the functionalized polynucleotide as described herein. The container optionally includes one or more additional type of surfaces which have a sequence complementary to one or more sequences of a second target polynucleotide, or a second portion of the same target polynucleotide. The sicPN is also optionally labeled with a detectable label as disclosed herein.

In another embodiment, the kit comprises at least two containers. The first container holds one or more surfaces as disclosed herein having one or more polynucleotides as described herein attached thereto which have a sequence complementary to one or more sequences of a target polynucleotide. The second container holds one or more surfaces having one or more polynucleotides attached thereto which have a sequence complementary to one or more sequences of the same or a different portion of the target polynucleotide, or to a second target polynucleotide.

In another embodiment, the kits have polynucleotides and surfaces in separate containers, and the polynucleotides are functionalized to the surfaces prior to use for detecting and/or inhibiting a target polynucleotide. In one aspect, the polynucleotides and/or the surfaces are functionalized so that the polynucleotides can be attached to the surfaces. Alternatively, the polynucleotides and/or surfaces are provided in the kit without functional groups, in which case they must be functionalized prior to performing the assay.

### EXAMPLES

### Example 1

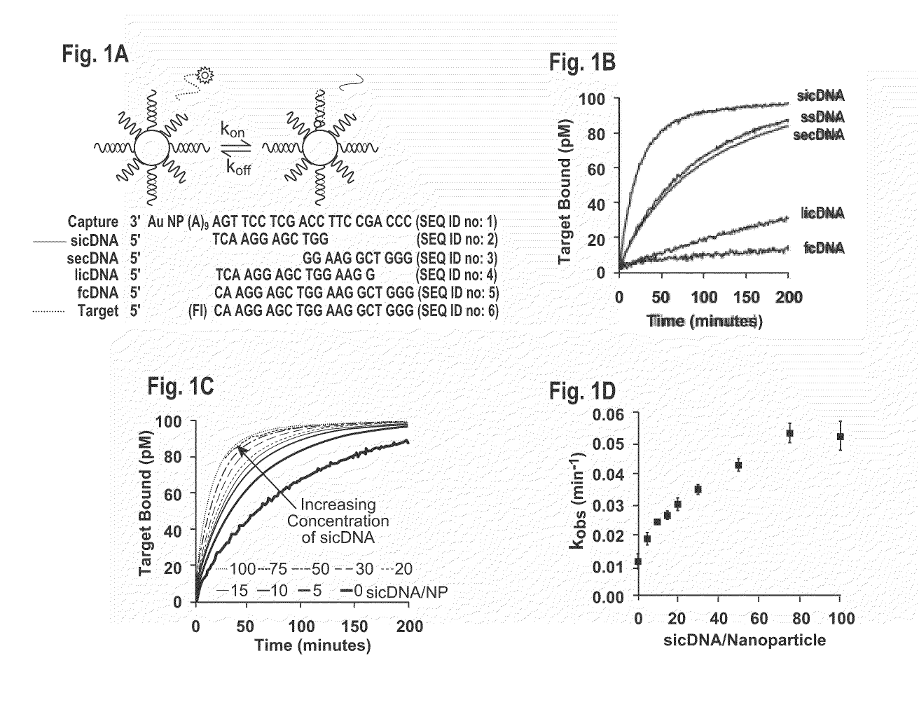

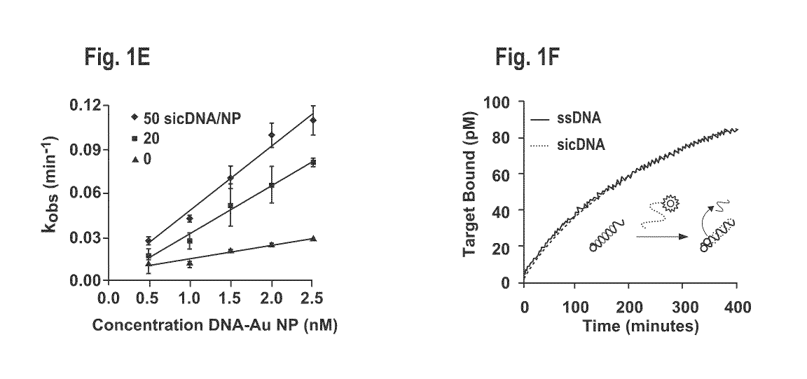

Au NPs (13±1 nm) were synthesized by citrate reduction of HAuCl₄ and were subsequently functionalized with DNA containing a 3′ propylthiol-A₁₀ spacer and a 5′ 20 base-pair recognition region [Hurst et al., Anal. Chem. 78: 8313-8318 (2006)]. DNA-Au NP syntheses and basic characterizations were preformed according to published methods [Hurst et al., Anal. Chem. 78: 8313-8318 (2006); Seferos et al., ChemBioChem 8: 1230-1232 (2007)]. After purification from excess polynucleotides, there were on average 73±18 DNA strands per nanoparticle as determined using a commercial DNA concentration assay [Seferos et al., ChemBioChem 8: 1230-1232 (2007)]. Complementary DNA was then hybridized to the DNA-Au NPs. The efficient distance-dependent quenching of the gold surface was used to monitor the hybridization rate of DNA-Au NPs with fluorophore-labeled targets. The rate of target hybridization to the DNA-Au NPs was measured with ssDNA-Au NPs or with DNA-Au NPs in the presence of one of four unlabeled complements (short internal complement (sicDNA), short external complement (secDNA), long internal complement (licDNA), and full complement (fcDNA)) (FIG. 1a). In these experiments, the labeled target and the unlabeled complement can both bind to the same region of DNA. In contrast to previous kinetic experiments [Riccelli et al., Nucleic Acids Res. 29: 996-1004 (2001); Yuan et al., Chem. Commun. 6600-6602 (2008); Vasiliskov et al., Nucleic Acids Res. 29: 2303-2313 (2001); O'Meara et al., Anal. Biochem. 255: 195-203 (1998); Maye et al., J. Am. Chem. Soc. 128: 14020-14021 (2006)], this prohibits simultaneous binding of sicDNA and target to the same capture strand, preventing any additional base-stacking interactions from occurring. Binding rates were determined using fluorescence measurements recorded on a Jobin Yvon Fluorolog FL3-22. Complementary target DNA (100 pM) was added to DNA-Au NPs (1 nM) in phosphate buffered saline with Tween 20 (0.1%), and the change in fluorescence over time was measured in 1 minute increments. Fluorescein (excitation=490 nm, emission=520 nm) and Cy5 (excitation 633 nm, emission=670) were monitored at their respective wavelengths. k_{obs} for each binding curve was determined by fitting the data to simple association kinetics equation ([Target Bound]=[Target Bound]ₘₐₓ*e^{k}_{obs}*^{time}). kₒₙ and k_{off} were determined using a linear fit (kₒₙ=(k_{obs}-k_{off})/[Nanoparticles]).

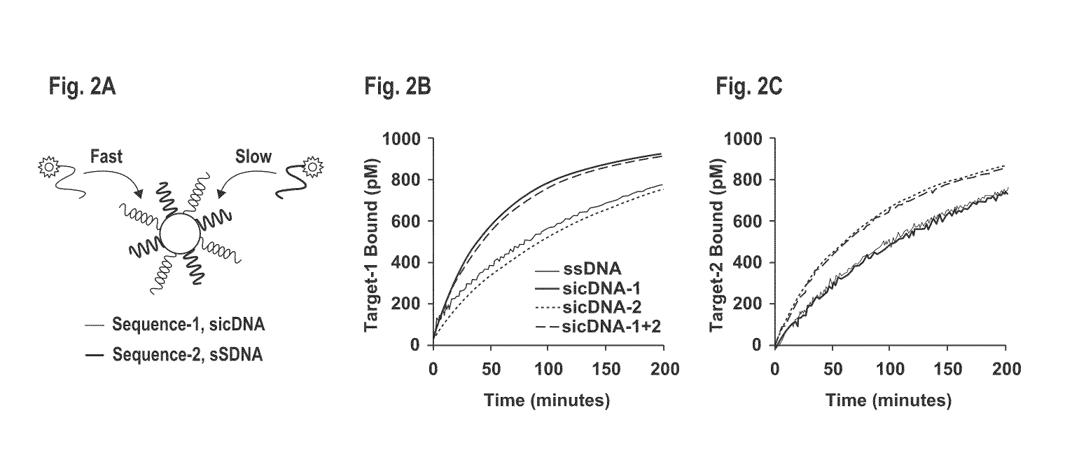

As expected, long unlabeled complements (licDNA and fcDNA) act as competitive inhibitors, greatly reducing the observed rate of association (k_{obs}=0.0020±0.0001 and 0.0008±0.0002 min⁻¹ respectively) when compared to ssDNA-Au NPs (0.011±0.002 min⁻¹) (FIG. 1b). Since sicDNA also binds in the target hybridization site, one might expect it to act as a competitive inhibitor as well, however, this DNA architecture actually increases rate of target binding (k_{obs}=0.030±0.002 min⁻¹) when compared to ssDNA-Au NPs. Similar experiments were repeated with other DNA sequences and LNA-DNA chimera. In all cases, a significant rate enhancement was observed when sicDNA was used (FIG. 2), indicating that this is a general strategy for increasing the hybridization rate, and is applicable to a wide range of DNA designs. Additionally, the effect of complement position was investigated by measuring the rate of target hybridization in the presence of short external duplexes with the same predicted binding strength as the sicDNA. Unlike internal complements, external complements have no significant effect on hybridization rate (k_{obs}=0.010±0.002 min⁻¹) (FIG. 1b), indicating that leaving an external ssDNA binding site available for incoming target is critical for the rate enhancement observed with sicDNA. Together, these results show that although most unlabeled complements inhibit the hybridization of a labeled target, the use of sicDNA results in more rapid binding. In particular, the combination of an internal dsDNA region with a relatively long ssDNA region (approximately 9 base pairs) appears to be important for the rate increase.

### Example 2

The next investigation was to look at k_{obs} as a function of the number of sicDNA strands per Au NP. In all experiments, the number of available binding sites on the DNA-Au NPs was in excess of the number of target molecules in solution. Under these conditions, the reaction reached equilibrium with approximately 100% of the target bound [Lytton-Jean et al., J. Am. Chem. Soc. 127: 12754-12755 (2005)]. The rate of association increased as a function of sicDNA concentration (FIG. 1c). The hybridization rate hit a plateau as the concentration of sicDNA approached the concentration of DNA covalently bound to the Au NP, reaching a maximum k_{obs} that was 4.7-fold more rapid than a ssDNA-Au NP (FIG. 1d), consistent with a rate enhancing DNA structural change at the nanoparticle surface.

In order to further characterize the kinetics of the system, the relative contribution of kₒₙ and k_{off} to the change in k_{obs} was investigated. These parameters were calculated for nanoparticles at three different concentrations of sicDNA (0, 20, and 50 sicDNA/NP) (FIG. 1e). Conjugates with 50 sicDNA strands per nanoparticle were found to have 5-fold more rapid kₒₙ than ssDNA-Au NPs (kₒₙ=0.009±0.004, 0.033±0.004, and 0.045±0.003 nM⁻¹*min⁻¹, respectively for the 3 conditions). No significant difference in k_{off} was observed for any of the DNA-Au NPs under these conditions.

### Example 3

This example was performed to determine if the increased association rate was due to the DNA structure alone or a cooperative event involving the combined architecture of the DNA strands immobilized on a surface. The rate of DNA hybridization in the absence of Au NPs was measured using a molecular quencher in place of the gold nanoparticle. Kinetic measurements showed that the addition of sicDNA caused no observable change in rate when the DNA was not bound to the particle surface (FIG. 10, which is consistent with previous studies using similar DNA structures [Turberfield et al., Phys. Rev. Lett. 90: 118102 (2003); Gidwani et al., Analyst 134: 1675-1681 (2009)]. From this was concluded that the sicDNA-induced rate enhancement is a cooperative property, arising from the combined architecture of the sicDNA and the nanoparticle surface. This distinguished sicDNA from previous techniques for increasing hybridization kinetics, because neither hairpin disruption [Seelig et al., J. Am. Chem. Soc. 128: 12211-12220 (2006); Wei et al., Nucleic Acids Res. 36: 2926-2938, (2008); Gao et al., Nucleic Acids Res. 34: 3370-3377 (2006); Zhang et al., J. Am. Chem. Soc. 131: 17303-17314 (2009); Wang et al., Phys. Rev. E 72: 051918 (2005); Leunissen et al., Nat. Mater. 8: 590-595 (2009); Dreyfus et al., Phys. Rev. Lett. 102: 048301 (2009)] nor base-stacking [Riccelli et al., Nucleic Acids Res. 29: 996-1004 (2001); O'Meara et al., Anal. Biochem. 255: 195-203 (1998); Maye et al., J. Am. Chem. Soc. 128: 14020-14021 (2006)] mechanisms are surface-specific.

### Example 4

The sicDNA-Au NP conjugates contain two distinct types of binding sites: sicDNA-bound sites and unbound ssDNA sites. The origin of this rate increase could involve altering the DNA conformation specifically on the sicDNA-bound strand, thereby increasing the hybridization rate at that site. Alternately, sicDNA could change the conformation of DNA globally across the nanoparticle surface, thereby increasing the rate at both sicDNA-bound and unbound ssDNA sites. To distinguish these possibilities, Au NPs were functionalized with two different DNA sequences, each with its own sicDNA and target, creating a mixed-monolayer of DNA on the nanoparticle surface (FIG. 2a). If sicDNA increases the binding rate to all nanoparticle-bound DNA, one would expect the addition of a single sicDNA sequence to increase the hybridization rate for both targets on the same nanoparticle. However, the results of the experiments showed that sicDNA specifically increased the hybridization rate for its corresponding target and had no effect on the other target sequence (FIG. 2b-c). In order to increase the hybridization rate of both targets simultaneously, both sicDNAs were required. This observation suggested that targets bind sicDNA sites preferentially over ssDNA sites on the nanoparticle surface. Additionally, this demonstrated that one can selectively “turn on” the target binding kinetics for a specific sequence, even in a mixed monolayer of DNA, allowing for applications in complex, multi-component systems.

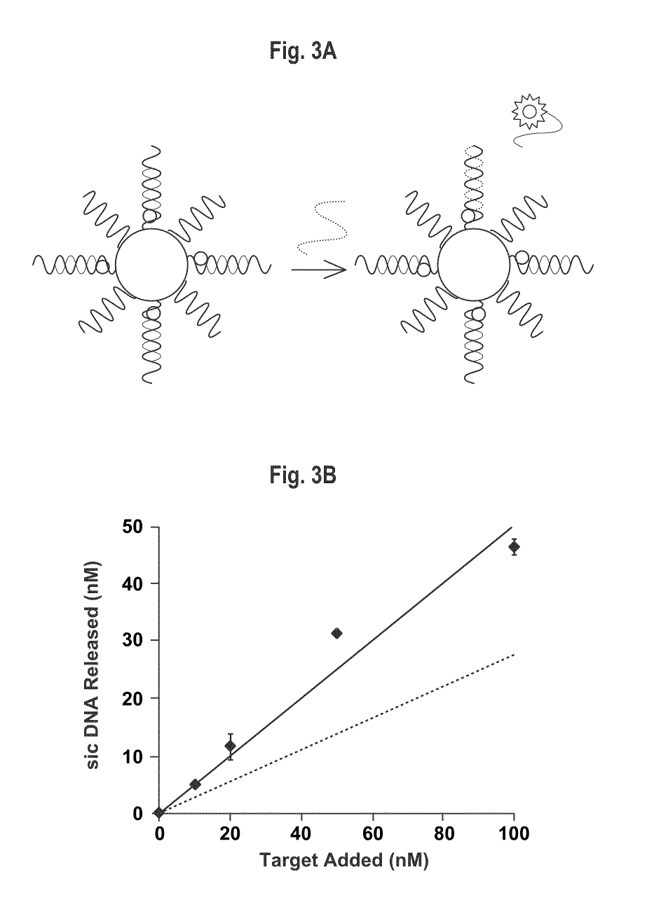

The displacement of sicDNA is consistent with the finding that the target is associating directly with sicDNA-bound sites. However, because ssDNA sites are also available for target binding, it is possible that the majority of sicDNA is not displaced upon target binding, but rather moves to an adjacent open site. To measure the release of sicDNA, rather than the association of target, sicDNA was labeled with a fluorophore, and its release was monitored by fluorescence after the addition of unlabeled target DNA (FIG. 3a). The final concentration of sicDNA released was determined as a function of target concentration and compared to a value calculated using the assumption that no sicDNA enhanced hybridization occurs (FIG. 3b). If target binding causes sicDNA reorganization to an adjacent ssDNA site then total sicDNA release would be relatively low. However, in these experiments sicDNA was preferentially released from the nanoparticle surface, consistent with both the preferential target hybridization at these sites and selective sicDNA release. This indicated that sicDNA cannot easily jump from strand-to-strand along the nanoparticle surface, but rather created a kinetically favored binding site on the nanoparticle surface and was released upon target association. These experiments showed the importance of sicDNA-based rate enhancement in the design of intracellular mRNA detection and regulation agents such as, for example and without limitation, the nanoflare.

### Example 5

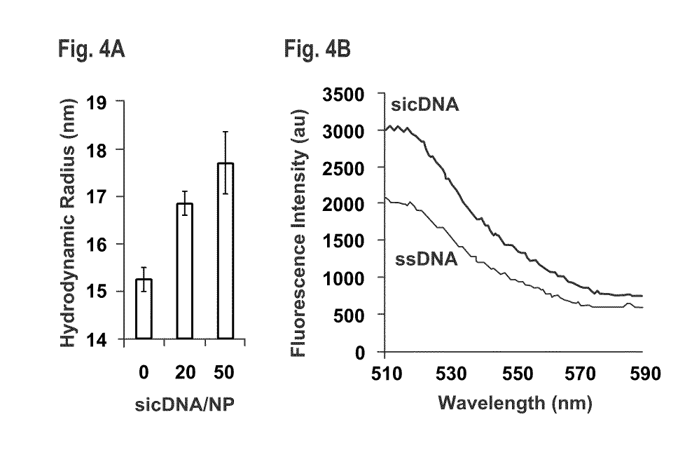

To determine if sicDNA had an effect on the overall structure of the DNA-Au NPs, dynamic light scattering (DLS) was used to measure the hydrodynamic radii of the nanoparticles (FIG. 4a). The hydrodynamic radius of DNA-Au NPs (1 nM) was measured by DLS before and after the addition of sicDNA (Zetasizer Nano ZS, Malvern). At high loadings of sicDNA, the radius increased by as much as 2.5±0.9 nm, consistent with a sicDNA-induced structural change. Although the DLS experiment provided information about the general DNA-Au NP structure, specific regions of the DNA appeared to be particularly important to increase the hybridization rate. A relatively long region of ssDNA must be present on the external end of the DNA-Au NP (FIG. 1b). To directly investigate the position of this external DNA region, the DNA covalently bound to the nanoparticle surface was labeled on the distal end with a fluorophore, and the nanoparticle-associated fluorescence was measured before and after the addition of sicDNA strands. When sicDNA was added to the DNA-Au NPs an increase in fluorescence was observed which was attributed to an increase in the distance between the distal DNA end and the nanoparticle surface [Dubertret et al., Nat. Biotechnol. 19: 365-370 (2001); Stoermer et al., J. Am. Chem. Soc. 128: 13243-13254 (2006); You et al., Nat. Nanotechnol. 2: 318-323 (2007); Maxwell et al., J. Am. Chem. Soc. 124: 9606-9612 (2002); Dulkeith et al., Nano Lett. 5: 585-589 (2005); Lee et al., J. Phys. Chem. C 113: 2316-2321 (2009)] (FIG. 4b). This result, taken with the observed change in nanoparticle radius (FIG. 4a) and the requirement for a distal ssDNA region in the sicDNA architecture (FIG. 1), showed that sicDNA acts by extending the ssDNA region away from the Au NP surface, making it more available to incoming targets.

### Example 6

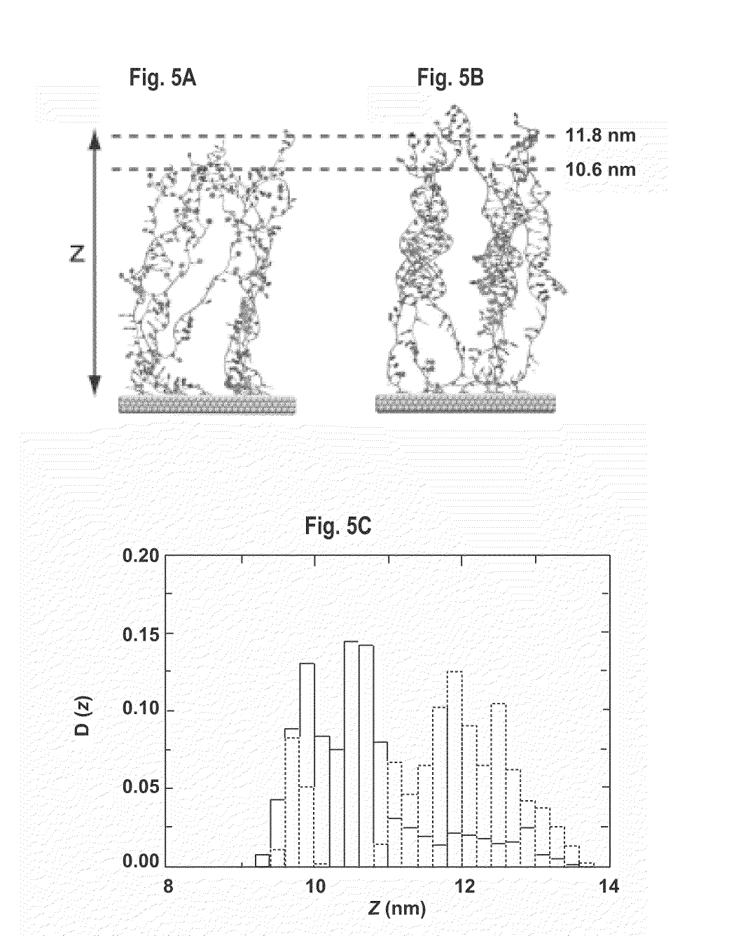

The sicDNA induced change in surface architecture was further investigated through Molecular Dynamics (MD) simulations. A flat gold surface was modeled with either seven ssDNA (FIG. 5a) or seven sicDNA (FIG. 5b) strands bound. When the sicDNA is present the distance between the terminal base and the gold surface is increased by 1.2±1.3 nm (FIG. 5c), within one standard deviation of the increase measured by DLS. The modeling results, taken with the experimental structural studies (FIG. 4), establish that sicDNA causes a conformational change, increasing the height of the DNA monolayer and moving the terminal ssDNA region away from the particle surface. This agrees with previous simulations of DNA on gold surfaces [Lee et al., J. Phys. Chem. C 113: 2316-2321 (2009)] and showed that the movement of the terminal ssDNA region increases its availability for target binding, thereby causing the observed increase in target binding rate.

The increase in hybridization rate that was observed with sicDNA-Au NPs is contemplated to be general to a wide range of surface-based DNA technologies. The role of DNA density was investigated, because many of the unique cooperative binding properties of the DNA-Au NPs [Lytton-Jean et al., J. Am. Chem. Soc. 127: 12754-12755 (2005); Giljohann et al., Nano Lett. 7: 3818-3821 (2007)] arise because the DNA monolayer is much more dense than on traditional flat surfaces. To test if density plays a role in hybridization kinetics, DNA-Au NPs were created with 85% fewer DNA strands per nanoparticle, compared to the DNA-Au NPs described above [Giljohann et al., Nano Lett. 7: 3818-3821 (2007)]. The k_{obs} for target binding on the sparsely functionalized nanoparticles was much higher in the presence of sicDNA, indicating that high DNA density is not critical for the sicDNA-based rate increase.

To further investigate the generality of sicDNA-induced binding rate increases, analogous binding rate experiments were performed on microarrays. The microarrays containing ssDNA and sicDNA spots were prepared on Codelink slides (SurModics) according to the manufacturer's recommendations. Fluorophore labeled target was incubated with the microarray in 0.2×SSC (30 mM NaCl, 3 mM sodium citrate, pH 7) with 0.1% sodium dodecylsulfate, and the reaction was stopped by washing and drying the slides. Slides were imaged using a Zeiss 510 LSM microscope (10× magnification) and a 488 nm Argon laser. Fluorescence was quantified using ImageJ.

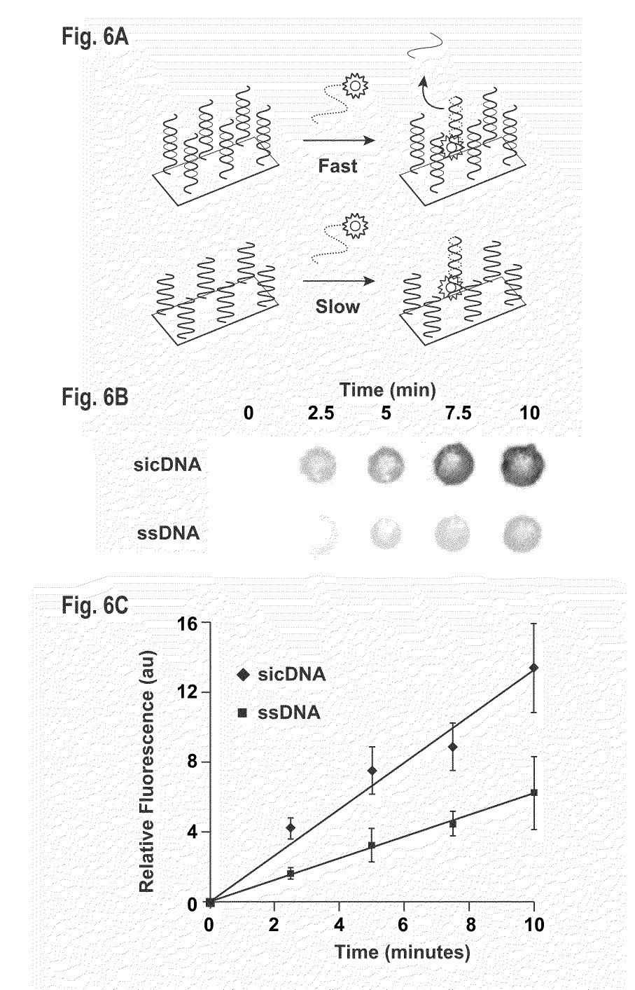

A microarray was created with ssDNA and sicDNA spots in different locations (FIG. 6a). Fluorophore-labeled target DNA was hybridized to the chip, the fluorescence associated with each spot was quantified as a function of time, and the initial hybridization rate was determined (FIG. 6b). A 2-fold increase in initial binding rate was observed with sicDNA. Although the increase was not as great as observed on the DNA-Au NPs, this result still confirmed that sicDNA is used to increase the rate of hybridization on both high and low DNA density surfaces, and thus, is compatible with a wide range of applications, including intracellular detection, gene regulation, and microarrays.

Due to its nanoscale structure and its dynamic and controllable target binding properties, DNA plays an increasingly important role in a wide range of fields and in the development of new technologies. Control of DNA hybridization kinetics in the context of functional devices and materials are needed for continued improvement and growth in this area. sicDNA induces changes to DNA surface structure, resulting in the presentation of an external ssDNA site that can easily initiate target binding and increases the overall rate of target hybridization. By adding specific sicDNA sequences, the binding of a target is selectively “turned on,” even in the presence of multiple sequences and targets. sicDNA increased the rate of target hybridization for all the nucleic acids and surfaces tested, including microarrays and DNA-Au NPs, which are powerful reagents for intracellular experiments such as mRNA detection [Seferos et al., J. Am. Chem. Soc. 129: 15477-15479 (2007)] and gene regulation [Rosi et al., Science 312: 1027-1030 (2006)].

### Example 7

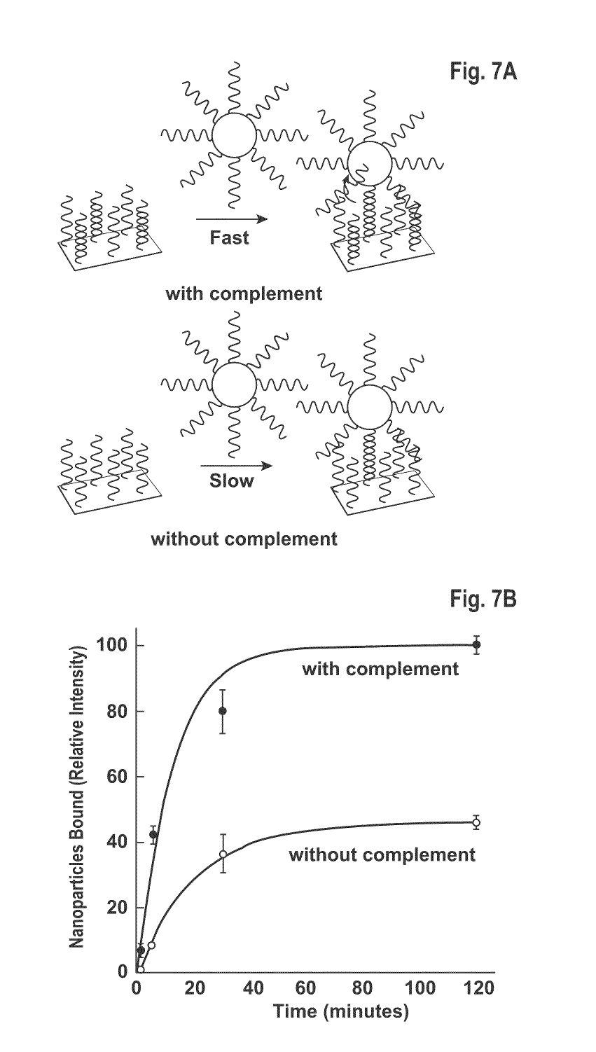

This example shows that short-displaceable complement polynucleotides enhance binding rate on glass slides. Codelink slides were functionalized with DNA in the presence or absence of complement polynucleotide. They were then incubated with gold nanoparticles and the target nucleic acid. The binding was measured by silver staining, followed by light-scattering analysis. In samples containing the displaceable duplex a stronger signal rapidly appeared (FIG. 7).

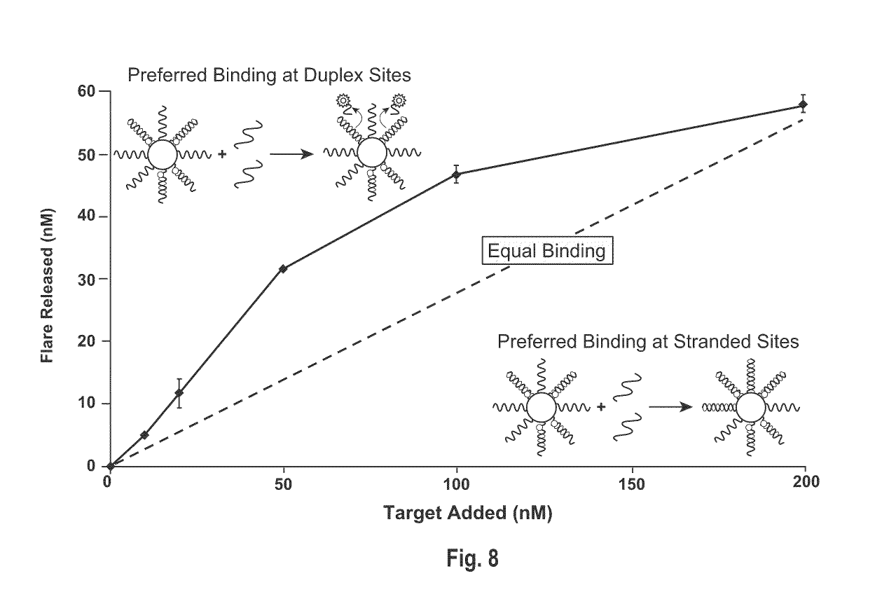

This example also shows that displacement complements are released from a surface in response to target binding. This was measured fluorescently for nanoparticles containing 20 complement polynucleotides and 50 single stranded DNA polynucleotides per nanoparticle. The large release of complement polynucleotide (FIG. 8) indicates enhanced association of target with complement bound sites.

1. A composition comprising a surface functionalized with a plurality of polynucleotides, each polynucleotide in the plurality functionalized to the surface at a terminus of the polynucleotide, the composition further comprising
a plurality of short internal complementary polynucleotides (sicPNs) having a sequence sufficiently complementary to a portion of each polynucleotide in the plurality such that under appropriate conditions, a sicPN in the plurality of sicPNs is able to associate with each polynucleotide over a portion of each polynucleotide;
the portion of each polynucleotide located proximal to the terminus of each polynucleotide that is functionalized to the surface;
each polynucleotide having a length longer than each sicPN in the plurality to provide a single stranded portion of each polynucleotide when a polynucleotide in the plurality is associated with a sicPN in the plurality of sicPNs, the single stranded portion of the polynucleotide located distal to the portion of the polynucleotide to which the sicPN associates, the single stranded portion having a sequence sufficiently complementary to a target polynucleotide to associate with the target polynucleotide under appropriate conditions, wherein association of the polynucleotide with the target polynucleotide displaces and/or releases the sicPN associated with the polynucleotide; and
wherein at least 25% of all polynucleotides in the plurality are associated with a sicPN.

2. The composition of claim 1 wherein the surface is a nanoparticle or the surface is a solid support.

3. (canceled)

4. The composition of claim 2 wherein the solid support is a microarray.

5. The composition of claim 1, wherein association of the single stranded portion of the polynucleotide with the target polynucleotide causes a detectable change.

6. The composition of claim 5 wherein the sicPN comprises a detectable label that causes the detectable change when the target polynucleotide is associated with the single stranded portion or wherein the target polynucleotide comprises a detectable label that causes the detectable change when the target polynucleotide is associated with the single stranded portion.

7. (canceled)

8. The composition of claim 6 wherein the detectable label is a fluorophore.

9. The composition of claim 8 wherein the fluorophore is quenched when the sicPN is associated with a polynucleotide.

10. (canceled)

11. The composition of any of claim 1 wherein at least 75% of all polynucleotides are associated with a sicPN.

12. The composition of claim 1 wherein the single stranded portion of the polynucleotide is at least about 2 nucleotides to about 100 nucleotides in length.

13. The composition of claim 1 wherein rate of association between the polynucleotide and the target polynucleotide is increased when a sicPN is associated with the polynucleotide compared to rate of association between the polynucleotide and the target polynucleotide in the absence of the sicPN.

14. The composition of claim 13 wherein the association rate is increased by at least about 2-fold to at least about 5-fold.

15. The composition of claim 1 wherein the plurality of polynucleotides are each sufficiently complementary to a target polynucleotide to allow association.

16. The composition of claim 1 wherein each polynucleotide in the plurality of polynucleotides all have the same sequence or wherein at least two polynucleotides in the plurality of polynucleotides have different sequences.

17. (canceled)

18. The composition of claim 16 wherein polynucleotides that have different sequences each have different single stranded portions that associate with different target polynucleotides.

19. The composition of claim 18 wherein the different single stranded portions associate with the same target polynucleotide at different locations on the target polynucleotide or wherein the different single stranded portions associate with different target polynucleotides.

20. (canceled)

21. The composition of claim 16 wherein at least two sicPNs in the plurality of sicPNs have different sequences and each of the two sicPNs associate with different polynucleotides.

22. A method of detecting a target polynucleotide comprising contacting the target polynucleotide with the composition of claim 1, wherein contact between the target and the composition results in a detectable change.

23. A method of inhibiting expression of a gene product encoded by a target polynucleotide comprising contacting the target polynucleotide with a composition of claim 1 under condition sufficient to inhibit expression of the gene product.

24. The method of claim 23 wherein expression of the gene product is inhibited in vivo or is inhibited in vitro.

25. (canceled)

26. The method of claim 23 wherein the expression is inhibited by at least about 5%.


SEQUENCE LISTING (6 sequences)

SEQ ID NO: 1 - DNA, 30 bp, Artificial Sequence
  Synthetic primer
cccagccttccagctccttgaaaaaaaaaa

SEQ ID NO: 2 - DNA, 12 bp, Artificial Sequence
  Synthetic primer
tcaaggagctgg

SEQ ID NO: 3 - DNA, 11 bp, Artificial Sequence
  Synthetic primer
ggaaggctggg

SEQ ID NO: 4 - DNA, 16 bp, Artificial Sequence
  Synthetic primer
tcaaggagctggaagg

SEQ ID NO: 5 - DNA, 20 bp, Artificial Sequence
  Synthetic primer
caaggagctggaaggctggg

SEQ ID NO: 6 - DNA, 20 bp, Artificial Sequence
  Synthetic primer
caaggagctggaaggctggg